(19) United States
(12) Patent Application Publication
(10) Pub. No.: US 2021/0368892 A1
(43) Pub. Date: Dec. 2, 2021

(54) UNDERPANTS FOR PATIENT WITH BEDSORES

(71) Applicant: NATIONAL DEFENSE MEDICAL CENTER, Taipei City (TW)

(72) Inventors: En MENG, Taipei City (TW); Juin-Hong CHERNG, Taipei City (TW); Tien-Show LIANG, Taipei City (TW); Shu Ting LIANG, Taipei City (TW); Shu-Jen CHANG, Taipei City (TW); Shu Han LIANG, Taipei City (TW); Yi-Wen WANG, Taipei City (TW); Gang-Yi FAN, New Taipei City (TW)

(21) Appl. No.: 17/329,867

(22) Filed: May 25, 2021

(30) Foreign Application Priority Data
Jun. 2, 2020 (TW) 109206846

(51) Int. Cl. A41D 13/12; A41B 9/00; A41D 31/10; A41D 31/12

(52) U.S. Cl. CPC A41D 13/1254; A41D 31/12; A41D 31/10; A41B 9/001

(57) ABSTRACT

The present invention is related to a pair of underpants for patients with bedsores. The underpants include an underpants body and a patch. The underpants body includes a front half portion, a rear half portion opposite to the front half portion, and a crotch portion corresponding to the genital of the patient with bedsores and connected to the front half portion and the rear half portion to form a lumbar opening and two leg openings. The patch includes a fabric layer, an absorbent layer and a waterproof layer. The absorbent layer attaches to the fabric layer, while the waterproof layer attaches to the absorbent layer. The waterproof layer is adapted to attach to an inside surface of the underpants body so that the fabric layer may contact with the bedsore parts of the patient and thus absorb the moisture of the bedsores.

### CROSS-REFERENCES TO RELATED APPLICATIONS

This application claims priority to Taiwan Patent Application No. 109206846 filed on Jun. 2, 2020, the disclosures of which are incorporated by reference herein in their entirety.

### BACKGROUND OF THE INVENTION

### Field of the Invention

The present invention relates to a pair of underpants, and in particularly, a pair of underpants for patients with bedsores.

### Descriptions of the Related Art

Bedsores are caused by excessive pressure, shear or friction on the skin, and will cause necrosis of the subcutaneous tissue due to poor blood circulation. It is likely to occur in patients who have been bedridden for a long time due to injury or illness, and more commonly occurs in the protrusions of bones that are in contact with the support plane. Once the skin begins to ulcerate and form a gap, germs can easily enter the human body from the wound, causing more serious infection symptoms, and even life threatening. Since bedsores are difficult to cure, once they occur, it is extremely important to take care of them carefully to avoid deterioration.

In order to prevent bedsore wounds from being contaminated with bacteria, gauze is usually covered after the wound is cleaned to isolate the air and control the humidity of the wound, or to apply topical medications to more serious wounds to promote tissue regeneration. However, attaching gauze or adhesive tape directly onto or around the wound may easily tear the wound during the removal and cause discomfort to the patient.

When being bedridden or relying on a wheelchair for a long time, protrusions of bones in the hip area of the human body are prone to bedsores. In view of this, there is a great need for a kind of underpants that is convenient for caring for wounds in this field.

### SUMMARY OF THE INVENTION

An object of the present invention is to provide a pair of underpants for patient with bedsores (hereinafter referred to as “underpants”). A patch may be disposed thereon to attach to the bedsore parts of the patient with bedsores to absorb moisture from the bedsores.

To achieve the above object, the underpants of the present invention comprises an underpants body and at least one patch. The underpants body comprises a front half portion, a rear half portion opposite to the front half portion, and a cotch portion corresponding to the genital of the patient with bedsores. The front half portion and the rear half portion are connected together to form a lumbar opening and two leg openings. The at least one patch comprises a fabric layer, an absorbent layer and a waterproof layer, wherein the absorbent layer is attached to the fabric layer, the waterproof layer is attached to the absorbent layer, and the waterproof layer is attached to an inner side surface of the underpants body, so that the fabric layer is attached to the bedsore parts of the patient with bedsores to absorb moisture of the bedsores when the underpants are in use.

Another object of the present invention is to provide underpants for a patient with bedsores, comprising an underpants body. The underpants body is formed by a fabric layer, an absorbent layer and a waterproof layer, wherein the absorbent layer is attached to the fabric layer, the waterproof layer is attached to the absorbent layer. The waterproof layer comprises a front half portion, a rear half portion opposite to the front half portion, and a crotch portion corresponding to the genital of the patient with bedsores and connected to the front half portion and the rear half portion. The front half portion and the rear half portion are connected together to form a lumbar opening and two leg openings. The fabric layer and the absorbent layer have a shape corresponding to the waterproof layer, and the fabric layer is attached to skin of the patient with bedsores to absorb moisture of the bedsores when the underpants are in use, the waterproof layer is configured to exposure to external air.

Another object of the present invention is to provide underpants for a patient with bedsores, comprising an underpants body and at least one patch. The underpants body is formed by outer cloth and inner cloth corresponding to the outer cloth. The underpants body comprises a front half portion, a rear half portion opposite to the front half portion, and a crotch portion corresponding to the genital of the patient with bedsores and connected to the front half portion and the rear half portion, wherein the front half portion and the rear half portion are connected together to form a lumbar opening and two leg openings, the outer cloth and the inner cloth are connected to define an accommodating space, and the inner cloth further comprises at least one opening. The at least one patch, disposed in the accommodating space and corresponding to the opening, the patch comprises a fabric layer, an absorbent layer and a waterproof layer, wherein the absorbent layer is attached to the fabric layer, the waterproof layer is attached to the absorbent layer, and a central area of the fabric layer is exposed from the opening, so that the fabric layer is attached to the bedsore parts of the patient with bedsores to absorb moisture of the bedsores.

In an embodiment, the patch of the underpants of the present invention further comprises an adhesive, disposed on a surface periphery of the fabric layer, so that the patch is removably attached to an outer side of the inner cloth, and completely covers the opening. The adhesive can also disposed on a surface of the waterproof layer, so that the patch is removably attached to an inner side of the outer cloth, and completely covers the opening.

In an embodiment, a lower edge of the underpants body of the underpants of the present invention is positioned between the lower hip and knees of the patient with bedsores, while an upper edge of the underpants body covers iliac crest parts of the patient with bedsores.

To make the above objects, technical features and advantages more obvious and easier to understand, detailed description of preferred embodiments, along with the accompanying drawing are provided below.

### BRIEF DESCRIPTION OF THE DRAWINGS

FIG. 1 is a schematic view of the bedsore frequent occurrence positions of human body;

FIG. 2 is a schematic perspective view of the underpants of a first preferred embodiment according to the present invention;

FIG. 3 is an exploded schematic perspective view of the patch of the underpants of FIG. 2;

FIG. 4A to FIG. 4D are schematic views of a variety of shapes of patches disposed on the underpants of FIG. 2;

FIG. 5 is an exploded perspective schematic view of the underpants of a second preferred embodiment according to the present invention;

FIG. 6 is an exploded perspective schematic view of the underpants of a third preferred embodiment according to the present invention; and

FIG. 7A to FIG. 7B are exploded perspective schematic views of the patch of the underpants of FIG. 6.

### DESCRIPTION OF THE PREFERRED EMBODIMENT

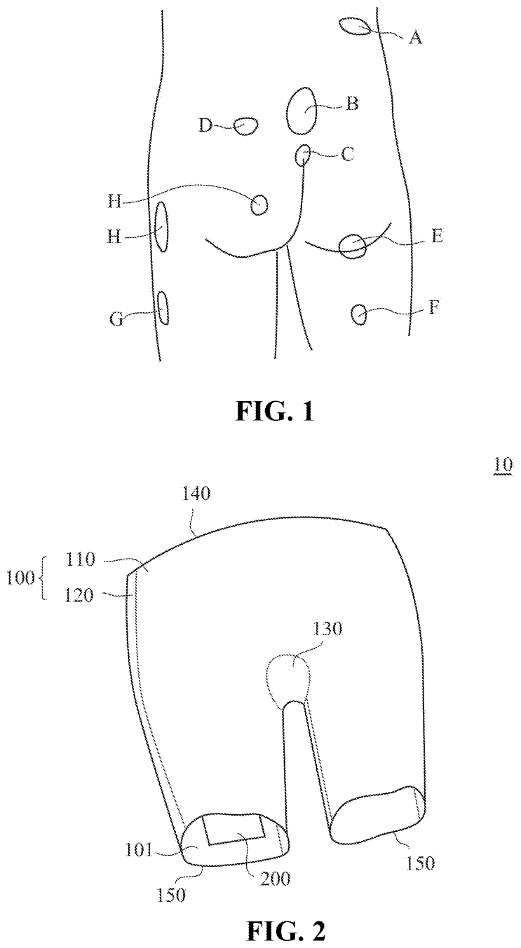

FIG. 1 is a schematic view of the bedsore frequent occurrence positions of human body. The bedsore frequent occurrence positions include iliac crest part A, sacral part B, coccyx part C, sacroiliac part D, ischial part E, posterior thigh F, side thigh G and buttock and hip H, etc.

FIG. 2 is a schematic view of the underpants of a first preferred embodiment according to the present invention. The underpants 10 comprises an underpants body 100 and a patch 200, the technical contents of each element is describe as followed.

Underpants body 100 comprises a front half portion 110, a rear half portion 120 opposite the front half portion 110, and a crotch portion 130. The front half portion 110 may correspond to the lower abdomen and part of the anterior thighs of patients with bedsores, the rear half portion 120 may correspond to buttock and hip and part of posterior thigh, and the crotch portion 130 correspond to the genital of the patient with bedsores and connects to the front half portion and the rear half portion 120. As the front half portion 110 and the rear half portion 120 are connected, a lumbar opening 140 is formed on the upper edge and two leg openings 150 are formed on the lower edge. Underpants body 100 may be formed by an elastic fiber, so that, when the patient is wearing underpants 10, the underpants body 100 may attach onto the patient's skin, and also cooperate with different cutting and stitching methods to make the underpants body 100 do not have excessive pressure (for example, no more than 32 mmHg) on the bedsore frequent occurrence positions to avoid the deterioration of bedsores.

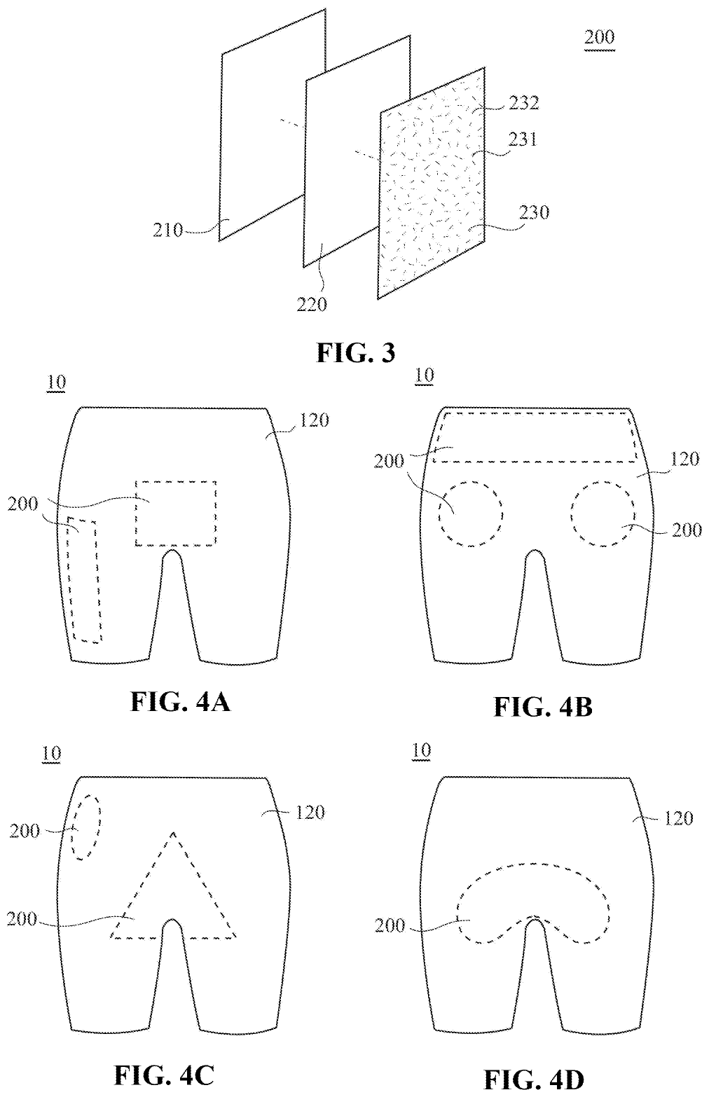

Please refer to FIG. 3, it's an exploded perspective schematic view of the patch 200. The patch 200 comprises a fabric layer 210, an absorbent layer 220 and a waterproof layer 230. A side of the absorbent layer 220 is attached to the fabric layer 210, the waterproof layer 230 is attached to the water absorbent layer 220, the waterproof layer 230 may further comprises an adhesive 231, disposed on the surface 232 of the other side of the waterproof layer 230, so that the patch 200 may be removably attached to the inner side surface 101 of the underpants body 100, and thereby, the fabric layer 210 may be attached to the bedsore parts of the patient with bedsores to absorb moisture of the bedsores when the underpants are in use.

In detail, the fabric layer 210 may be formed by non-woven fabrics, cotton and/or other materials to reduce friction when it is pressed against the bedsore parts, reducing the chance of secondary injury to the wound, and reduce the discomfort of the patient. The absorbent layer 220 may be formed by foam, seaweed gel and/or other materials, to absorb moisture from the bedsores, control the humidity of the wound and improve healing effect. The waterproof layer 230 may be a plastic to prevent the moisture from the absorbent layer 220 leaking out and contaminate the underpants body 100. The adhesive 231 is capable of repeated pasting, having sufficient adhesion on the fabric, in this way, the patient who attaches the patch 200 to the underpants body 100 may adjust and change the sticking position of the patch 200 multiple times to match the patient's bedsores position.

Correspond to the bedsore frequent occurrence positions shown by FIG. 1, a lower edge of the underpants body 100 of the underpants 10 (at the leg openings 150) is preferred to position between the lower hip and knees of the patient with bedsores, an upper edge of the underpants body 100 (at the lumbar opening 140) is preferred to position at iliac crest parts of the patient with bedsores, to make the range of attaching of the patch 200 more flexible. As shown in FIG. 4A to FIG. 4D, the patch 200 may be disposed on multiple positions of the underpants body 100 according to different bedsore parts, and may have a shape of rectangle, square, circle, polygon, or others, to better fit the curvature of the body of the bedsore parts, preventing a gap between the fabric layer 210 and the bedsore parts that let the moisture which cannot be absorbed efficiently leak out along the gap. Take a rectangle patch as example, it is preferred to have a variety of sizes of 8×7.5 cm and 8×15 cm.

Next, the underpants 20 of the second preferred embodiment of the present invention will be described. Part of the technical contents of the underpants 20 and the technical contents of the underpants 10 can be referred to each other, and the same parts will be omitted or simplified.

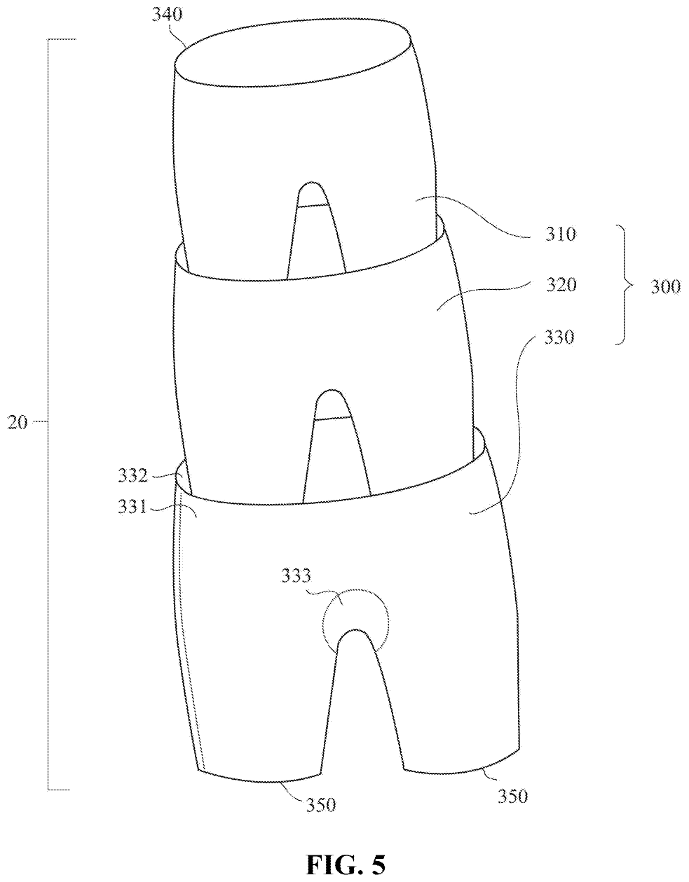

Please refer to FIG. 5, it is an exploded perspective schematic view of the underpants 20 of the second preferred embodiment of the present invention. Generally, the underpants 20 is a pants-like patch, comprising an underpants body 300 having a fabric layer 310, an absorbent layer 320 and a waterproof layer 330. The waterproof layer 330 comprises a crotch portion 333 corresponding to the genital of the patient with bedsores, a front half portion 331 and a rear half portion 332. The front half portion 331 and the rear half portion are connected together, to form a lumbar opening 340 and two leg openings 350. The fabric layer 310 and the absorbent layer 320 may have a shape corresponding to the waterproof layer 330. An upper edge of the underpants body 300 is preferred to be positioned at the iliac crests parts of the patient with bedsores, and the lower edge is preferred to be positioned between the lower hip and knees of the patient with bedsore. In this way, when the underpants 20 are in use, the fabric layer 310 may be pressed against to all the lower abdomen area, part of the thigh area, buttock and hip, and crotch area, to absorb moisture of the bedsores in a large area for patients with more serious bedsores, without attaching too many patches 200 to the inner surface 101 of the underpants body 100.

Next, the underpants 30 of the third preferred embodiment of the present invention will be described. Part of the technical contents of the underpants 30 and the technical contents of the underpants 10 can be referred to each other, and the same parts will also be omitted or simplified.

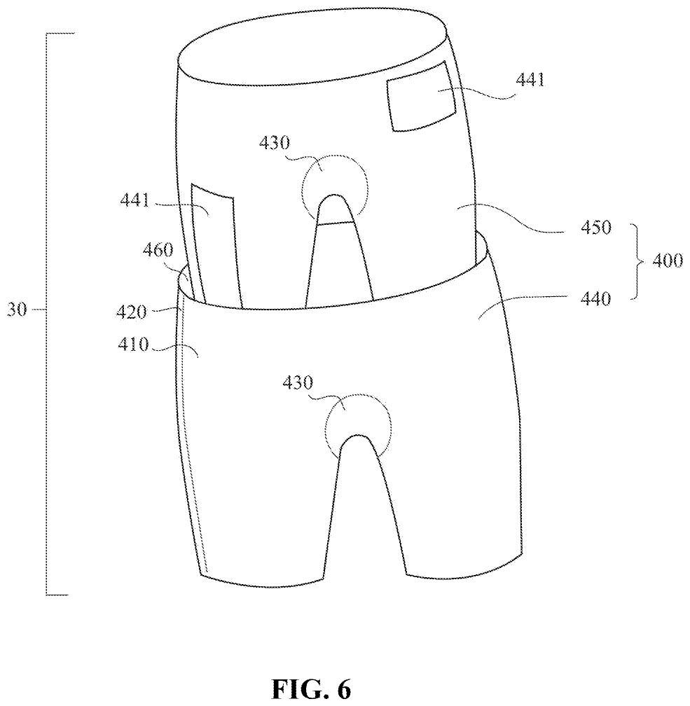

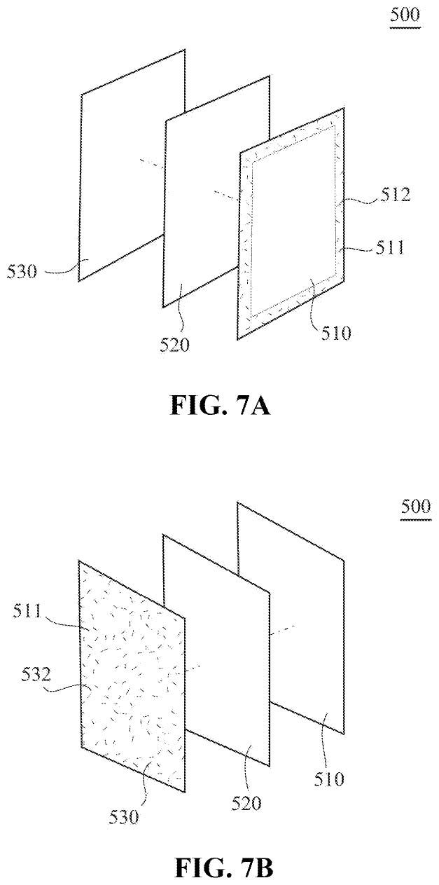

Please refer to FIG. 6 to FIG. 7B at the same time, the underpants 30 comprises underpants body 400 and a patch 500, the structure of the underpants body 400 is like the underpants body 100, having a front half portion 410, a rear half portion 420 and a crotch portion 430. The difference is that the underpants body 400 is formed by outer cloth 440 and inner cloth 450 corresponding to the outer cloth 440. The outer cloth 440 and the inner cloth 450 can be sewn and connected to each other to define an accommodating space 460. The inner cloth 450 has at least one opening 441 which may be positioned at multiple bedsore frequent occurrence positions as FIG. 4A to FIG. 4B has shown.

The structure of the patch 500 is like the patch 200, having a fabric layer 510, an absorbent layer 520 and a waterproof layer 530. The difference is that it may be disposed in the accommodating space 460 and correspond to the opening 441. In detail, the patch 500 may comprises an adhesive 511, disposed on a surface periphery 512 of the fabric layer 510, so that the fabric layer 510 of the patch 500 is correspond to the opening 441, removably attached to an outer side of the inner cloth 450, and completely covers the opening 441, such that the fabric layer 510 may abut to bedsore parts of the patient with bedsores (the adhesive 511 will not contact with the patient with bedsores). Alternatively, the adhesive 511 may also be disposed on a surface 532 of the waterproof layer 530 of the patch 500, so that the patch 500 may be removably attached to an inner side of the outer cloth 440, and completely correspond to the opening 441. In this way, when the underpants 30 are in use, the fabric layer 510 of the patch 500 may attach to bedsore parts of the patient with bedsores, to absorb the moisture of the bedsores.

Accordingly, a patch may be flexibly disposed within the underpants of the present invention, to attach onto the bedsore parts of the patient with bedsores, to absorb the moisture from the bedsores and to prevent tearing the wounds during the removal.

The above embodiments are only for exemplifying the implementation of the present invention, and to explain the technical features of the present invention, and are not for limiting the protection scope of the present invention. Any modifications or equivalence arrangements that can be easily completed by those familiar with the art are within the scope of the present invention. The scope of protection of the rights of the present invention shall be subject to the scope of the claims.

What is claimed is:

1. A pair of underpants for a patient with bedsores, comprising:
an underpants body, comprising a front half portion, a rear half portion opposite to the front half portion, and a crotch portion corresponding to the genital of the patient with bedsores and connected to the front half portion and the rear half portion, wherein the front half portion and the rear half portion are connected together to form a lumbar opening and two leg openings; and
a patch, comprising a fabric layer, an absorbent layer and a waterproof layer, wherein the absorbent layer is attached to the fabric layer, the waterproof layer is attached to the absorbent layer, and the waterproof layer is attached to an inner side surface of the underpants body, so that the fabric layer is attached to the bedsore parts of the patient with bedsores to absorb moisture of bedsores when the underpants are in use.

2. The underpants of claim 1, wherein the patch further comprises an adhesive disposed on a surface of the waterproof layer, so that the patch is removably attached to the inner side surface of the underpants body.

3. The underpants of claim 1, wherein a lower edge of the underpants body is positioned between the lower hip and knees of the patient with bedsores.

4. The underpants of claim 1, wherein an upper edge of the underpants body covers iliac crest parts of the patient with bedsores.

5. A pair of underpants for a patient with bedsores, comprising:
an underpants body, comprising a fabric layer, an absorbent layer and a waterproof layer, wherein the absorbent layer is attached to the fabric layer, the waterproof layer is attached to the absorbent layer;
wherein the waterproof layer comprises a front half portion, a rear half portion opposite to the front half portion, and a crotch portion corresponding to the genital of the patient with bedsores and connected to the front half portion and the rear half portion;
wherein the front half portion and the rear half portion are connected together to form a lumbar opening and two leg openings, shapes of the fabric layer and the absorbent layer are corresponding to the waterproof layer, and the fabric layer is attached to skin of the patient with bedsores to absorb moisture of bedsores when the underpants are in use, the waterproof layer is configured to exposure to external air.

6. The underpants of claim 5, wherein an upper edge of the underpants body covers iliac crest parts of the patient with bedsores, and a lower edge of the underpants body is positioned between the lower hip and knees of the patient with bedsores.

7. A pair of underpants for a patient with bedsores, comprising:
an underpants body, formed by outer cloth and inner cloth corresponding to the outer cloth, wherein the underpants body comprises a front half portion, a rear half portion opposite to the front half portion, and a crotch portion corresponding to the genital of the patient with bedsores and connected to the front half portion and the rear half portion, wherein the front half portion and the rear half portion are connected together to form a lumbar opening and two leg openings, the outer cloth and the inner cloth are connected to define an accommodating space, and the inner cloth further comprises at least one opening; and
a patch, disposed in the accommodating space and corresponding to the opening, the patch comprises a fabric layer, an absorbent layer and a waterproof layer, wherein the absorbent layer is attached to the fabric layer, the waterproof layer is attached to the absorbent layer, and a portion of the fabric layer is exposed from the opening, so that the fabric layer is attached to the bedsore parts of the patient with bedsores to absorb moisture of bedsores.

8. The underpants of claim 7, wherein the patch further comprises an adhesive disposed on a surface periphery of the fabric layer, so that the patch is removably attached to an outer side of the inner cloth, and completely covers the opening.

9. The underpants of claim 7, wherein the patch further comprises an adhesive disposed on a surface of the waterproof layer, so that the patch is removably attached to an inner side of the outer cloth, and completely covers the opening.

10. The underpants of claim 7, wherein an upper edge of the underpants body covers iliac crest parts of the patient with bedsores, and a lower edge of the underpants body is positioned between legs and knees of the patient with bedsores.
